(19) United States
(12) Patent Application Publication
(10) Pub. No.: US 2007/0259903 A1
(43) Pub. Date: Nov. 8, 2007

(54) ANTI-VIRAL USES FOR ANALOGS OF BARBITURIC ACID

(76) Inventor: Kirpal S. Gulliya, Carmel, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/691,092

(22) Filed: Mar. 26, 2007

Related U.S. Application Data
(60) Provisional application No. 60/788,080, filed on Apr. 1, 2006.

(51) Int. Cl. A61K 31/515; A61K 31/513

(52) U.S. Cl. 514/269; 514/270

(57) ABSTRACT

A method of using certain barbituric acid analogs to kill RNA viruses, including Group IV and Group V viruses such as SARS and influenza viruses. The barbituric acid analog may have the structure (S1) shown below: 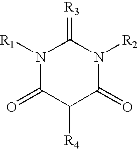
where R₁ and R₂ are independently hydrogen, C₁-C₆ alkyl, C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O, S, Se or C(CH₃)₂; and R₄ is hydrogen. One particularly preferred barbituric acid analog has the structure (S1) shown above where R₁ and R₂ are both butyl, R₃ is S, and R₄ is hydrogen. The barbituric acid analogs may be used in vivo, such as in birds or in humans or other animals, or they may be used ex vivo, such as in air handling systems or on hard surfaces. The barbituric acid analogs may be incorporated into animal or bird feed or supplements. Particularly effective compositions of barbituric acid analogs dissolved in DMSO are also disclosed.

### RELATION TO RELATED APPLICATIONS

This application claims the benefit of U.S. Provisional Patent Application Ser. No. 60/788,080, filed Apr. 1, 2006, the entire contents of which are hereby incorporated herein by reference.

### FIELD OF THE INVENTION

The present invention relates generally to the use of a family of barbituric acid analogs to treat viruses, and more particularly to the use of certain barbituric acid analogs to treat RNA viruses such as influenza viruses and specific strains thereof.

### BACKGROUND OF THE INVENTION

The synthesis of barbituric acid starting from hydurilic and diliuric acid was first accomplished by Baeyer (1864). Barbital, a derivative of barbituric acid, was prepared by Conrad and Guthzeit (1882). The pharmacological properties of barbital were later described (Fisher and Mering, 1903; Baeyer, F. & Co., Ger. Pat. 247952, Mar. 04, 1911). The barbiturates act as nonselective central nervous system depressants and are primarily used as sedative hypnotics and anti-convulsants in subhypnotic doses (Myer and Rollet, 1964). For example, the sodium salts of amobarbital, pentobarbital, phenobarbital, and secobarbital are presently available as prescription drugs. It is important to note that the basic structure common to these drugs, barbituric acid, in itself has no central nervous system activity. Central nervous system activity is obtained by substituting certain alkyl, alkenyl or aryl groups on the pyrimidine ring structure. The role of gamma-aminoisobutyric acid on anticonvulsant activity has been described (Hafeley, 1980).

Only a few derivatives of thiobarbituric acid were prepared until 1935, when it was discovered that these compounds possess anesthetic properties (Miller et al., 1935; Tabern and Volwiler, 1935; Miller et al., 1936). Derivatives with a thiosemicarbazide group at the C-5 position and phenylhydrazones of 5-monoalkylbarbituric acids display antimicrobial properties (Kamel, 1982; Chemishev, 1979; Chemishev, 1981). The presence of allyl or n-decyl groups also confers antimicrobial properties (Beres et al., 1980; Beres et al., 1974). Certain barbituric acid derivatives are photooxidation products of Merocyanine 540, and are reportedly useful as anticancer and antiviral agents (U.S. Pat. No. 5,312,919). In addition, certain N-substituted barbituric acids with halogen atoms at C-5 display antiviral properties, including influenza virus (Ger. Offen 2003994, Belg. Pat. 622081).

In addition to the above, the inventor of the present invention discovered in the 1990s that certain barbituric acid analogs provide anti-cancer and anti-pathogenic properties. U.S. Pat. Nos. 5,674,870 and 5,869,494 (incorporated herein by reference), among others, resulted from that work. While anti-viral properties were generally suggested in that earlier work, only efficacy against DNA viruses such as herpes virus, and against reverse transcribing viruses such as HIV viruses, were shown. Efficacy against RNA viruses, and particularly against influenza viruses and specific influenza strains, was neither disclosed nor suggested.

The classification of viruses commonly distinguishes between DNA viruses, RNA viruses, and reverse transcribing viruses. For example, the Baltimore classification is a classification system which places viruses into one of seven groups depending on a combination of their nucleic acid (DNA or RNA), strandedness (single-stranded or double-stranded), and method of replication.

With this system DNA viruses are broken into two groups, particularly:

Group I: viruses possess double-stranded DNA and include such virus families as Herpesviridae (examples like HSV1 (oral herpes), HSV2 (genital herpes), VZV (chickenpox), EBV (Epstein-Barr virus), CMV (Cytomegalovirus)), Poxviridae (smallpox) and many tailed bacteriophages. The mimivirus was also placed into this group; and

Group II: viruses possess single-stranded DNA and include such virus families as Parvoviridae and the important bacteriophage M13.

With the Baltimore classification system, RNA viruses are broken into three groups, particularly:

Group III: viruses possess double-stranded RNA genomes, e.g. rotavirus. These genomes are always segmented;

Group IV: viruses possess positive-sense single-stranded RNA genomes. Many well known viruses are found in this group, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus; and

Group V: viruses possess negative-sense single-stranded RNA genomes. The deadly Ebola and Marburg viruses are well known members of this group, along with influenza virus, measles, mumps and rabies.

Finally, with the Baltimore classification system reverse transcribing viruses are broken into two groups, particularly:

Group VI: viruses possess single-stranded RNA genomes and replicate using reverse transcriptase. The retroviruses are included in this group, of which HIV is a member; and

Group VII: viruses possess double-stranded DNA genomes and replicate using reverse transcriptase. The hepatitis B virus can be found in this group.

Influenza viruses belong to the genus orthomyxovirus in the family Orthomyxoviridae, and are classified as a Group V (RNA) virus. Influenza viruses are commonly classified on the basis of the antigenicity of the nucleoprotein or matrix protein into three main groups: Influenza A, Influenza B, and Influenza C.

Influenzavirus A has only one species in it, namely, the species commonly called “Influenza A virus.” Influenza A virus causes “avian influenza” (also known as bird flu, avian flu, Influenzavirus A flu, type A flu, or genus A flu), and is commonly hosted by birds. It is also believed to potentially infect several species of mammals, and potentially may be passed to humans.

Flu strain H1N1 is a subtype of the Influenza A virus. H1N1 has mutated into various strains including the Spanish Flu strain (now extinct in the wild), mild human flu strains, endemic pig strains, and various strains found in birds. A variant of H1N1 is believed to have been responsible for the Spanish flu pandemic that killed some 50 million to 100 million people worldwide between 1918 and 1919.

Flu strain H3N2 is another subtype of the influenza A virus. H3N2 viruses are known to infect humans and pigs, though in each species the virus has mutated into many strains. H3N2 exchanges genes for internal proteins with other influenza subtypes.

A more recently discovered flu strain H5N1 is another influenza A virus subtype, and is known to cause illness in humans and many other animal species. A bird-adapted strain of H5N1, called HPAI A(H5N1) for “highly pathogenic avian influenza virus of type A of subtype H5N1”, is the causative agent of H5N1 flu, commonly known as “avian influenza” or “bird flu”. It is endemic in many bird populations, especially in Southeast Asia, and is believed to be spreading globally. Most references to “bird flu” in recent media reports refer to this strain.

The annual flu (also called “seasonal flu” or “human flu”) kills an estimated 36,000 people in the United States each year. Flu vaccines are based on predicting which mutants of H1 N1, H3N2, H1 N2, and influenza B will proliferate in the next season. Separate vaccines are developed for the northern and southern hemispheres in preparation for their annual epidemics. In the tropics, influenza shows no clear seasonality. In the past ten years, H3N2 has tended to dominate in prevalence over H1N1, H1N2, and influenza B. Measured resistance to the standard antiviral drugs amantadine and rimantadine in H3N2 has increased from 1% in 1994 to 12% in 2003 to 91% in 2005.

It is well appreciated by persons skilled in the art that a need continues to exist for anti-viral agents that may effectively be used against RNA viruses generally, and against Group V viruses particularly, including against influenza viruses and specific strains thereof. That need includes, but is not limited to, anti-viral agents that may be used to treat human and/or birds or animals, or to treat inanimate objects such as food, animal feed or supplements, or as disinfectants or as part of sterilization processes for human, animal, or bird environments. The present invention addresses that need.

### SUMMARY OF THE INVENTION

In one aspect of the present invention there is provided a method of using certain barbituric acid analogs to kill RNA viruses (including Group IV and Group V viruses such as SARS and influenza viruses) in vivo, including the use of the barbituric acid analogs to treat humans, birds, or animals. In this aspect of the present invention a therapeutic amount of a barbituric acid analog (or an optical isomer or a pharmaceutically acceptable salt thereof) is administered to the person, bird, or animal.

The barbituric acid analog may have the structure (S1) shown below: 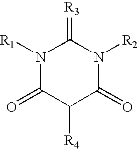
where R₁ and R₂ are independently hydrogen, C₁-C₆ alkyl, C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O, S, Se or C(CH₃)₂; and R₄ is H or O. In one particularly preferred embodiment the barbituric acid analogs have the structure (S1) shown above where R₁ and R₂ are both butyl, R₃ is S, and R₄ is hydrogen.

In another aspect of the present invention there is provided a method of using the barbituric acid analogs described above to kill RNA viruses ex vivo, such as in fluids (biological or otherwise), or air, or solids, or on hard or semi-hard surfaces.

### DESCRIPTION OF THE DRAWINGS

FIG. 1 shows a graph of the virucidal activity of C1 against H1N1.

FIG. 2 shows a graph of the virucidal activity of C1 against H3V2.

FIG. 3 shows a graph of the virucidal activity of C1 against H5N1.

### DESCRIPTION OF THE PREFERRED EMBODIMENTS

For the purposes of promoting an understanding of the principles of the invention, reference will now be made to certain embodiments and specific language will be used to describe the same. It will nevertheless be understood that no limitation of the scope of the invention is thereby intended, such alterations and further modifications of the illustrated embodiments being contemplated as would normally occur to one skilled in the art to which the invention relates.

As indicated above, the present invention provides a method for killing influenza virus by contacting the virus with a barbituric acid analog, or an optical isomer or a pharmaceutically acceptable salt thereof. In one aspect the invention provides methods for killing influenza A strains, including strains H1N1, H3N2, and H5N1. The viruses may be killed in vivo or ex vivo.

More particularly describing the barbituric acid analogs used in the present invention, the preferred barbituric acid analogs have the structure (S1) shown below: 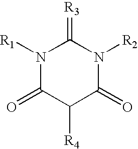
where R₁ and R₂ are independently hydrogen, C₁-C₆ alkyl, C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O, S, Se or C(CH₃)₂; and R₄ is H or O.

It is to be appreciated that the solubility and biodistribution of these compounds can be modified by varying the substituent R groups. Accordingly, in some preferred embodiments the barbituric acid analogs have the structure (S1) shown above where R₁ and R₂ are independently C₁-C₆ alkyl, R₃ is O, S, Se or C(CH₃)₂, and R₄ is hydrogen. In other preferred embodiments the barbituric acid analogs have the structure (S1) shown above where R₁ and R₂ are independently hydrogen, C₁-C₆ alkyl, C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O or S; and R₄ is hydrogen. In yet other preferred embodiments the barbituric acid analogs have the structure (S1) shown above where R₁ and R₂ are independently C₁-C₆ alkyl; R₃ is O or S; and R₄ is hydrogen.

In some preferred embodiments the barbituric acid analogs have the structure (S1) shown above where the C₁-C₆ alkyl substituent(s) are butyl, and in some of those preferred embodiments the barbituric acid analogs have the structure (S1) shown above where the C₁-C₆ alkyl substituent(s) are n-butyl. In one particularly preferred embodiment the barbituric acid analogs have the structure (S1) shown above where R₁ and R₂ are both butyl, R₃ is S, and R₄ is hydrogen.

In some embodiments the barbituric acid analogs have the structure (S1) shown above where R₁ and R₂ are independently hydrogen, C₁-C₆ alkyl, C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is S; and R₄ is hydrogen. In other embodiments the barbituric acid analogs have the structure (S1) shown above where the C₁-C₆ alkyl substituent(s) are n-butyl and R₃ is S.

In one particularly preferred embodiment of the barbituric acid analogs (referred to herein as compound C1), the analog has the structure (S1) shown above where R₁ and R₂ are both butyl, R₃ is S, and R₄ is hydrogen. In another particularly preferred embodiment of the barbituric acid analogs (referred to herein as compound C2), the analog has the structure (S1) shown above where R₁ and R₂ are both butyl, R₃ is S, and R₄ is oxygen. In yet another particularly preferred embodiment of the barbituric acid analogs (referred to herein as compound C4), the analog has the structure (S1) shown above where R₁ and R₂ are both hydrogen, R₃ is S, and R₄ is hydrogen. In still another particularly preferred embodiment of the barbituric acid analogs (referred to herein as compound C5), the analog has the structure (S1) shown above where R₁ and R₂ are both methyl, R₃ is S, and R₄ is hydrogen.

The compositions useful in the methods of the present invention may comprise a compound having the structure S1 as defined above, or they may comprise an optical isomer or a pharmaceutically acceptable salt of such compounds. The formulations may include a pharmaceutically acceptable carrier for intravenous, oral, or parenteral administration, or for ex vivo or in vitro use.

Preferred analogs of barbituric acid for the herein described methods are 4,6(1 H, 5H)-pyrimidinedione, 1,3-dibutyldihydro-2-thioxo, (compound Cl, which is structure S1 where R₁ and R₂ are CH₂CH₂CH₂CH₃, R₃ is S and R₄ is H); and 4,5,6(1H)-pyrimidinetrione, 1,3-dibutyldihydro-2-thioxo (compound C2, which is compound C1 having a doubly bonded oxygen at the 5 position).

In one aspect of the present invention there is provided a method of killing RNA virus, and particularly Group IV and/or Group V viruses such as SARS and influenza, by contacting the virus with an effective amount of a barbituric acid analog as described above. The virus may be contacted in vivo, in vitro, or ex vivo. In vivo treatment is useful for treating diseases in a body such as a human body, or an animal, such as a farm or a domestic animal or bird. In vitro treatment is useful, for example, for treating cells and tissues outside the body such as in blood or blood products or various forms of their suspensions or in cultures, including but not limited to the treatment of natural or cell derived component fluids found in human or animal body or synthetic fluids or growth hormones or supplements or culture medium, etc. Ex vivo treatment is particularly useful for killing virus on surfaces such as hard surfaces or semi-hard surfaces or incorporation, or coating or mixing or spraying of agent on such as but not limited to instruments or food, or animals or birds (domestic or wild) or animal feed or supplements or incorporating the agent in breathing masks for humans or air filtration devices for human or animal body.

For in vivo use, such as when treating birds, humans, or other animals, preferred therapeutic amounts are generally from about 0.001 mg/kg (of the bird or animal being treated) to about 3 g/kg. In other embodiments the barbituric acid analogs may be used at levels between about 0.01 mg/kg (of the bird or animal being treated) and about 1 g/kg.

When treating inanimate objects, or for other ex vivo use, the barbituric acid analog is preferably used at a concentration of from about 0.001 μg/ml to about 10 g/ml. In other embodiments the barbituric acid analogs may be used at concentrations between about 0.1 μg/ml to about 1 g/kg for ex vivo use.

One preferred method comprises the steps of dissolving a barbituric acid analog of the present invention in a pharmaceutically acceptable carrier and administering the formulation in a therapeutically effective amount to the body tissues or other surfaces or items that are, or may become associated or infected with influenza virus. A further preferred method includes oral administration of a barbituric acid analog of the present invention with or without first dissolving the compound in a pharmaceutically acceptable carrier. A pharmaceutically acceptable carrier may be in the form of a capsule, pill or as liquid, or metal or microbeads or slurry, or gel or spray or additive to food or supplements or lozenges or gum or nasal spray or mouth spray.

Thus, a solution of a barbituric acid analog of the present invention prepared in a pharmaceutically acceptable carrier and adjusted to the desired concentration may be used for either in vitro, in vivo, or ex vivo applications. For in vitro applications, a solution containing an effective amount of the therapeutic agent may be administered to body tissue or cells or body fluids or liquids, or suspensions, or hard or semi-hard surfaces, or incorporated in food, feed or supplements and the like outside the body (ex vivo), and, in particular, the body is a human or animal or bird body. The mixture containing a barbituric acid analog of the present invention and the tissues or cells to be treated are then incubated at an appropriate temperature for a desired duration of time. For in vivo applications, a barbituric acid analog of the present invention may be administered either orally, or rectally, or through skin, or a creme, lotion or spray, a solution containing an effective amount of the agent is directly administered into the animal body, for example, parenterally or by injection.

As used herein, the terms “contact”, “contacted”, and “contacting”, are used to describe the process by which an effective amount of a pharmacological agent, e.g., a compound provided by the present invention, is brought in direct juxtaposition with the target virus or a cell or both.

The term “body tissue” as used herein is to be understood to include “body fluid”, red blood cells, white blood cells, cryo precipitate from blood plasma, other plasma proteins, bone marrow, skin, cornea, organs and tissues from an animal or a human body, and the like.

The term “body fluids” as used herein is to be understood to include whole blood, any formed elements of the blood, blood plasma, serum, fluid containing such components, fluids from plasmapheresis, plasma fibrinogen, cryo-poor plasma, albumin, gamma globulins, semen, products of cells such as cytokines, hormones, growth or regulating factors and the like and other fluids introduced or intravenously, intramuscular or intra or sub-dermally injected into the body of a human or animal using known administration techniques. The term body fluid is to be understood to include body fluid prior to, or after, physical as well as chemical fractionation, separation or freezing or isolated or made from cells.

The term “external” or “ex vivo” as used herein is to denote outside the animal or human body.

The term “animal” as used herein is to denote a warm blooded animal including human, and domestic, wild and farm animals.

The phrase “chemomodifying agent” as used herein is to denote an agent, such as a chemical or any other agent, that can potentiate, augment or increase the therapeutic efficacy of a therapeutic agent. Hence, a chemomodifying agent can be an additive or a carrier, or a combination of products and may synergize the therapeutic efficacy of a therapeutic agent.

The phrase “a therapeutically effective amount” as used herein is to denote the concentration or quantity or level of the therapeutic agent that can attain a desired end, particular medical end, such as a treatment, or control or prevention or destruction of the undesirable virus, or cells, or cell-associated virus, or tumor cells or virus-infected cells, or pathogenic biological agent or contaminant, without producing unacceptable toxic symptoms.

The active compounds may be orally administered, for example, with an inert or acceptable diluent or with a carrier, or they may be enclosed in hard or soft shell gelatin capsule, or they may be compressed into tablets, or they may be incorporated directly with the food or supplements of the diet or delivered via a spray or chewing gum, etc. For oral therapeutic administration, the active compounds may be incorporated with excipients and used in the form of ingestible tablets, buccal tables, troches, capsules, elixirs, suspensions, syrups, wafers, time-release formulations, and the like. Such compositions and preparations should contain at least 0.01% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The term “carrier” as used herein denotes a vehicle, a solution containing water, buffer, ethanol, serum, serum proteins, lipoproteins, artificial bio-membranes, liposomes, monoclonal antibodies, carbohydrates, cyclodextrans, metal, microbeads, organic solvents, or other pharmaceutically acceptable or compatible solutions. The carrier, or vehicle, may or may not dissolve a barbituric acid analog of the present invention, and may enhance delivery of the therapeutic agent into effective proximity to the target such as a virus or virus infected cells or tumor cells or other pathogenic biological contaminants infecting the body or diseases of the immune system. The final carrier, or vehicle, used is pharmaceutically compatible in that it is relatively non-toxic to the normal cells and normal tissues. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compounds, sucrose as a sweetening agent, methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions or as attachments to antibody or microbeads or other compatible agents and sterile powders for the extemporaneous preparation of sterile injectable solutions or dispersions. In most cases, the form must be sterile and must be fluid to the extent that easy syringability exists. It must be stable under the conditions of manufacture and storage and must be preserved against the contaminating action of microorganisms, such as bacteria and fungi. The carrier can be a solvent, organic or inorganic, or dispersion or substrate medium containing, for example, water, metal, or protein (e.g. an antibody) or ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and other ingredients. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, or attachment vehicles such as microbeads, metallic or like substrate, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In one aspect of the present invention, the barbituric acid analogs (or optical isomers or salts thereof are used to kill RNA viruses (as classified by the Baltimore virus classification). In another aspect of the present invention, the barbituric acid analogs (or optical isomers or salts thereof) are used to kill RNA viruses that are classified in Group IV or Group V by the Baltimore virus classification. In yet another aspect of the present invention the barbituric acid analogs (or optical isomers or salts thereof) are used to kill influenza viruses, particularly including influenza A and/or influenza B viruses.

Reference will now be made to specific examples using the processes described above. It is to be understood that the examples are provided to more completely describe preferred embodiments, and that no limitation to the scope of the invention is intended thereby.

### EXAMPLE 1

### Synthesis of Compound C1: 4,6(1H, 5H)-Pyrimidinedione, 1,3-dibutyldihydro-2-thioxo

The subject compound was synthesized as described generally in Brown (1962) as follows: A 3-necked 2 liter flask fitted with a water condenser, gas inlet, and suba seal was purged with dry N₂. A freshly prepared solution of sodium ethyrate, prepared by slowly adding 2.5 g of Na to 50 ml EtOH at room temperature, was added to the flask. Diethyl malonate (17 g, 110 mmol) was then added with vigorous stirring, followed by the addition of N,N′-dibutyl-2-thiourea (10 g, 53 mmol). The mixture was stirred vigorously under N₂ for 3 days. The mixture was cooled to room temperature, 50 ml of water was added carefully and ethanol was removed under reduced pressure. The remaining residue was poured into water (200 ml) and cooled in an ice/water bath. The solution was filtered to remove unreacted starting materials and acidified with dilute HCl. The resulting precipitate was collected by suction filtration, washed with water, and dried thoroughly to give a white solid. The yield was about 75%. Compound C1 was determined to be approximately 98% pure by TLC and NMR.

### EXAMPLE 2

### Synthesis of Compound C2: 4,5,6(1H)-pyrimidinetrione, 1,3-dibutyldihydro-2-thioxo

A sample of N,N′-dibutyl-thiobarbituric acid (10 g) was dissolved in toluene (100 ml) and dried SeO₂ (10 g) was added. A steady stream of air was passed through the solution, and the solution was heated to reflux for 1 h. After cooling overnight, the solvent was removed under reduced pressure and the residue was chromatographed on activated alumina using chloroform as eluant. The desired compound was isolated after evaporation of the solvent and examined for purity by TLC and NMR. Yield-6 g.

### EXAMPLES 3-8

### Virucidal Activity Against Influenza Viruses

To help facilitate rapid development and commercialization of anti-viral and other therapeutic products the National Institute of Health (NIH) offers screening and evaluation services. At the direction of the present inventor virucidal testing was conducted by NIH for the purposes of assessing the efficacy of the compounds of the present invention against a panel of viruses generally, and particularly against influenza A and influenza B virus. The virucidal properties indicate efficacy against influenza in animal bodies, including humans, as well as inanimate uses such as sterilization of equipment or animal feed or addition to animal food or supplements, and a potential preventive agent in masks, air filtration devices and sprays, etc.

Compound C1 was tested as a blind sample against a panel of five different strains of RNA viruses, namely:

1. H1N1 (a strain of influenza A virus, commonly referred to as the New Caledonia strain);

2. H3N2 (a strain of influenza A virus, commonly referred to as the California strain);

3. H5N1 (a strain of influenza A virus, commonly referred to as the Vietnam strain);

4. A strain of influenza B virus commonly referred to as the Shanghai strain; and

5. A strain of Severe Acute Respiratory Syndrome (SARS) virus commonly referred to as the Urbani strain.

For each test, a stock solution of the lead Compound C1 was made by first dissolving it in a small amount (50-100 microliters) of absolute ethanol (ETOH) followed by addition of growth nutrient medium to a final volume of 1 ml to bring the stock concentration of 20 mg/ml. It was tested at a concentration of 100 μg/ml, with half-log dilutions down to 0.032 μg/ml.

The compound C1 and the virus being tested were incubated together at 8 different concentrations for 1 hour at 37° C. Then virus/compound mixture applied to established cultured MDCK (Madin-Darby canine kidney) cells for 1 hour at 37° C. After 1 hour, the Virus/compound were removed, and replaced with new virus/compound free medium, and the indicator cells were further incubated for 6 days. Virus that survived the one hour pre-incubation and one hour exposure to indicator cells caused cytopathic effect (CPE) development. Results were evaluated by microscopic inspection (visual) as well as a spectrophotometic Neutral Red Dye Assay. The values shown represent the concentration (μg/ml) of C1 that caused a 50% virucidal effect (EC50). The positive control (ribavirin) was not removed from cells.

The EC50 values were calculated by extrapolation from a plot of concentration versus virucidal activity data on semi-log paper. This allowed us to quantify complete virus killing of a small amount of virus (about 50-100 cell culture infectious doses).

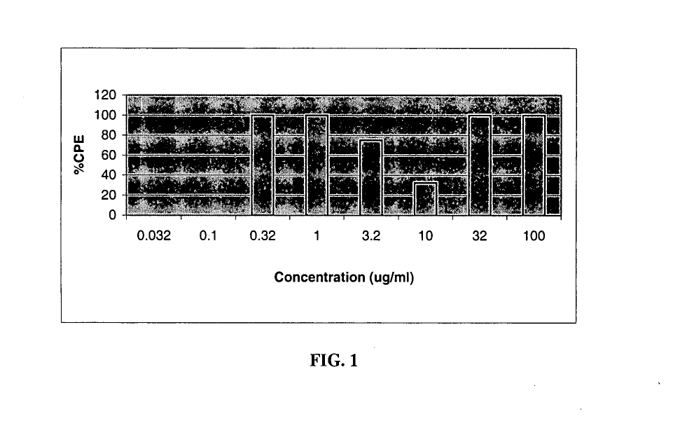

FIG. 1 shows a graph of the virucidal activity of C1 against H1N1. Flu A (H1N1) New Caledonia was treated with different doses of C1 for one hour at 37° C. Aliquots of treated and control virus samples were applied to the indicator cells. After one hour of incubation at 37° C. the virus/compound mixture was replaced with virus/compound free medium and the indicator cells were incubated at 37° C. After 6 days of incubation results as percent cytopathic effect (% CPE) were recorded. Results show that a treatment of H1N1 with C1 resulted in a virtually complete inactivation of the H1N1 virus. The dose that caused a 50% inactivation (EC50) was calculated to be 5.5 μg/ml by visual method. Therefore as little as 5.5 μg/ml of C1 was effective in destroying 50% of the Flu A (H1N1) virus.

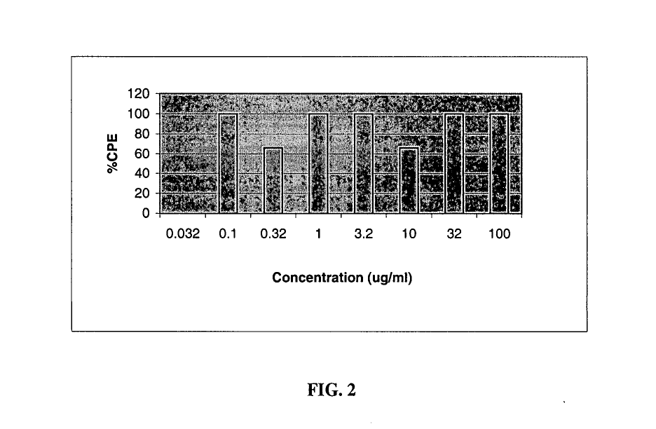

FIG. 2 shows a graph of the virucidal activity of C1 against H3V2. Flu A (H3N2) New Caledonia was treated with different doses of C1 for 4 hours. Aliquots of treated and control virus samples were applied to the indicator cells. After one hour of incubation at 37° C. the virus/compound mixture was replaced with virus/compound free medium and the indicator cells were incubated at 37° C. After 6 days of incubation results were recorded as percent cytopathic effect (% CPE). Results show that a treatment of H3N2 with C1 resulted in a virtually complete inactivation of the H3N2 virus. The dose that caused a 50% inactivation (EC50) was calculated to be 5.5 μg/ml by visual method.

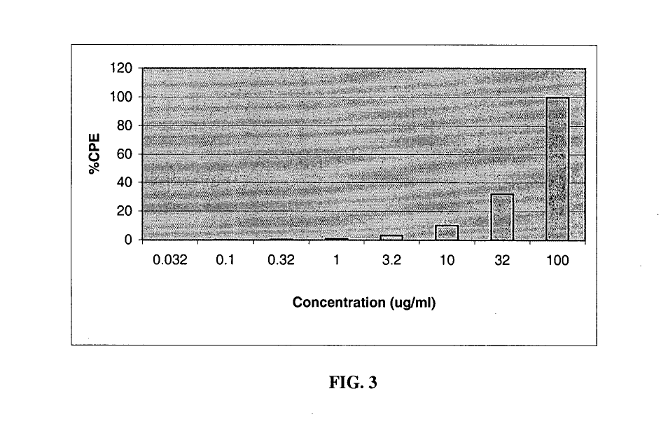

FIG. 3 shows a graph of the virucidal activity of C1 against H5N1. Flu A (H5N1) was treated with different doses of C1 for one hour. Aliquots of treated and control virus samples were applied to the indicator cells. After one hour of incubation at 37° C. the virus/compound mixture was replaced with virus/compound free medium and the indicator cells were incubated at 37° C. After 6 days of incubation results were recorded as percent cytopathic effect (% CPE). Results show that the dose that caused a 50% inactivation (EC50) was calculated to be 6.0 μg/ml visual method and 37 μg/ml by Neutral red assay. These data show that as little as a 2 hour treatment with C1 was effective in destroying the Flu A (H5N1) virus.

It can be seen from the above that C1 is an effective antiviral agent against all of the influenza A strains tested.

The treatment of Flu B Shanghai virus with C1 caused a 50% inactivation of the virus at a dose of 42 μg/ml by the visual method, and 37 μg/ml by Neutral red dye assay. The results indicate that Cl is an effective antiviral agent against Flu B virus.

The treatment of SARS (urbani), a Group IV virus with C1 caused a 50% inactivation of the virus at dose of 12 μg/ml by the visual method, and 53 μg/ml by Neutral Red dye assay. The results indicate that C1 is an effective antiviral agent against SARS.

### EXAMPLES 9-24

The antiviral activity of barbituric acid analogs was also evaluated under conditions where the indicator cells to which virus had been previously added and then continuously exposed to different concentrations of C1 for the duration of the experiments. Eight different doses of C1 ranging from 100 μg/ml to half-log dilutions down to 0.032 μg/ml were added to the indicator cells after adding the indicated virus. This virus-cell combination was continuously exposed to C1 until untreated control wells developed a cytopathic effect (CPE) usually after 4 to 6 days of incubation at 37° C. At the end of incubation period at 37° C., the resulting cytopathic effect (CPE) in control and test wells was assessed by visual and a Neutral Red Dye assay.

Results of CPE versus concentration of C1 were plotted on a semi-log paper and the dose of C1 that caused a 50% inhibition of virus mediated CPE (EC50) was calculated. The data show that barbituric acid analogs have anti-viral properties at doses ranging from 3 μg/ml to 40 μg/ml, as shown in Table 2 below. These results indicate that the subject analogs of barbituric acid are effective antiviral agents against RNA viruses.

TABLE 1
Antiviral Activity of Ananlogs of Barbituric Acid

| | | | | CELL | DRUG | |
|---|---|---|---|---|---|---|
| EXAMPLE | ASSAY | VIRUS | VIRUS STRAIN | LINE | UNITS | EC50 |
| 9 | Neutral | Flu A | New | MDCK | μg/ml | >3.2 |
| | Red | (H1N1) | Caledonia/20/99 | | | |
| 10 | Visual | Flu A | New | MDCK | μg/ml | 3.2 |
| | | (H1N1) | Caledonia/20/99 | | | |
| 11 | Neutral | Flu A | California/7/04 | MDCK | μg/ml | 3.2 |
| | Red | (H3N2) | | | | |
| 12 | Visual | Flu A | California/7/04 | MDCK | μg/ml | 3.2 |
| | | (H3N2) | | | | |
| 13 | Neutral | Flu A | Vietnam/1203/2004 | MDCK | μg/ml | 3 |
| | Red | (H5N1) | H | | | |
| 14 | Visual | Flu A | Vietnam/1203/2004 | MDCK | μg/ml | 3 |
| | | (H5N1) | H | | | |
| 15 | Neutral | Flu B | Shanghai/361/02 | MDCK | μg/ml | 3 |
| | Red | | | | | |
| 16 | Visual | Flu B | Shanghai/361/02 | MDCK | μg/ml | 3 |
| 17 | Neutral | Rhinovirus | HGP | HeLa | μg/ml | 30 |
| | Red | | | Ohio-1 | | |
| 18 | Visual | Rhinovirus | HGP | HeLa | μg/ml | 30 |
| | | | | Ohio-1 | | |
| 19 | Neutral | Measles | Chicago | CV-1 | μg/ml | 40 |
| | Red | | | | | |
| 20 | Visual | Measles | Chicago | CV-1 | μg/ml | 30 |
| 21 | Neutral | PIV | 14702 | MA-104 | μg/ml | 30 |
| | Red | | | | | |
| 22 | Visual | PIV | 14702 | MA-104 | μg/ml | 30 |
| 23 | Neutral | RSV A | A2 | MA-104 | μg/ml | 30 |
| | Red | | | | | |
| 24 | Visual | RSV A | A2 | MA-104 | μg/ml | 30 |

RSV= Respiratory Synsycitial virus;
PIV= Parainfluenza virus

It can be seen from the above that C1 is an effective antiviral agent against all of the RNA viruses tested, including Group IV and Group V viruses and particularly influenza virus strains.

### EXAMPLE 25

### Comparison of Dilution in ETOH to Dilution in DMSO

Compound C1 was made up in both EtOH and in DMSO at a concentration of 20 mg/ml. It was tested at a concentration of 100 μg/ml, with half-log dilutions down to 0.032 μg/ml. The compound was tested against two viruses under two different conditions. In one test the compound and the virus were incubated together for 1 hour at room temperature, and then were added to indicator cells, left on cell for 3 days during the virus replication period. In the other test the compound and the virus were incubated together for 1 hour at room temperature, and then were added to indicator cells for 1 hour, then removed and medium devoid of C1 and virus applied to the indicator cells. The two viruses used in the test were: 1) Influenza A/New Caledonia/20/99 (H1N1); and 2) Influenza A/California/7/04 (H3N2).

The virucidal activity of C1 dissolved in DMSO was surprisingly and significantly superior to C1 dissolved in ethanol. The results obtained by neutral red dye assay are expressed as EC50-50% virus inhibitory concentration in μg/ml are shown in the Table below.

TABLE 2
Comparison of the Virucidal Activity of C1
When Dissolved in Ethanol or in DMSO

| | | | Increase |
|---|---|---|---|
| | EC50 of C1 | EC50 of C1 | virucidal |
| Name of | in ETOH | in DMSO | activity of C1 |
| Virus | (μg/mL) | (μg/mL) | in DMSO |
| H1N1 | >4.5 | >2.6 | 200% |
| exposed to | | | |
| c1 + virus for | | | |
| 3 days | | | |
| H1N1 | 42.0 | 30.0 | 140% |
| exposed to | | | |
| c1 + virus for | | | |
| 1 hour | | | |
| H3N2 | >1.8 | >1.9 | 0% |
| exposed to | | | |
| c1 + virus for | | | |
| 3 days | | | |
| H3N2 | 42.0 | 20.0 | 220% |
| exposed to | | | |
| c1 + virus for | | | |
| 1 hour | | | |

While the compositions and methods of the present invention have been described by reference to certain preferred embodiments, it will be apparent to those of skill in the art that variations may be applied to the compositions and methods described herein without departing from the concept and spirit of the invention. All such modifications apparent to those skilled in the art are desired to be protected, and are deemed to be within the scope of the invention as herein disclosed and claimed.

What is claimed is:

1. A method for killing an RNA virus comprising contacting the RNA virus with a barbituric acid analog having the structure: 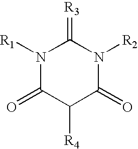
where R₁ and R₂ are independently hydrogen, C₁-C₆ alkyl C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O, S, Se or C(CH₃)₂; and R₄ is H or O; or an optical isomer or a pharmaceutically acceptable salt thereof.

2. The method of claim 1 wherein the RNA virus is a Group IV or a Group V virus.

3. The method of claim 2 wherein the Group IV or Group V virus is a SARS virus.

4. The method of claim 2 wherein the Group IV or Group V virus is an influenza virus.

5. The method of claim 4 wherein the influenza virus is an influenza A virus.

6. The method of claim 5 wherein the influenza A virus is H1N1.

7. The method of claim 5 wherein the influenza A virus is H3N2.

8. The method of claim 5 wherein the influenza A virus is H5N1.

9. The method of claim 5 wherein the influenza virus is an influenza B virus.

10. The method of claim 1 wherein said barbituric acid analog is a compound wherein R₁ and R₂ are both H or methyl or butyl, R₃ is S, and R₄ is H or O.

11. The method of claim 10 wherein said barbituric acid analog is a compound wherein R₁ and R₂ are both butyl and R₄ is H.

12. The method of claim 1 wherein said barbituric acid analog is dissolved in DMSO.

13. The method of claim 1 wherein the contacting is done in vivo.

14. The method of claim 1 wherein the contacting is done ex vivo.

15. The method of claim 1 wherein the contacting is done in liquids or solids, or on hard or semi-hard surfaces.

16. The method of claim 1 wherein the contacting is done by incorporating the barbituric acid analog in animal or bird feed or supplements, microbeads, or lozenges, or creme, or lotion, or chewing gum, or spray, or other treatment, or control or preventive device or agent.

17. The method of claim 1 wherein the contacting is facilitated by providing the barbituric acid analog in air or an air handling or filtration system.

18. A composition comprising a barbituric acid analog having the structure: 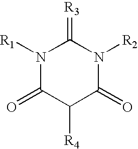
where R₁ and R₂ are independently hydrogen, C₁-C₆ alkyl C₁-C₆ alkoxy, C₁-C₆ haloalkyl, C₁-C₆ hydroxyalkyl, C₁-C₆ aminoalkyl, C₁-C₆ mercaptoalkyl, or aryl; R₃ is O, S, Se or C(CH₃)₂; and R₄ is H or O; or an optical isomer or a pharmaceutically acceptable salt thereof; wherein said barbituric acid analog or optical isomer or pharmaceutically acceptable salt thereof is dissolved in DMSO.

19. A composition according to claim 18 wherein said barbituric acid analog is a compound wherein R₁ and R₂ are both H or methyl or butyl, R₃ is S, and R₄ is H or O.

20. A composition according to claim 19 wherein said barbituric acid analog is a compound wherein R₁ and R₂ are both butyl and R₄ is H.
